(19) United States
(12) Patent Application Publication
(10) Pub. No.: US 2013/0030186 A1
(43) Pub. Date: Jan. 31, 2013

(54) PROCESS FOR PREPARING SULPHOXIDE COMPOUNDS

(71) Applicants: Vijay Chhangamal Chhabada, Baroda (IN); Arunkumar Gulabsingh Yadav, Baroda (IN); Rajeev Budhdev Rehani, Baroda (IN); Rajamannar Thennati, Baroda (IN)

(72) Inventors: Vijay Chhangamal Chhabada, Baroda (IN); Arunkumar Gulabsingh Yadav, Baroda (IN); Rajeev Budhdev Rehani, Baroda (IN); Rajamannar Thennati, Baroda (IN)

(73) Assignee: SUN PHARMACEUTICAL INDUSTRIES LIMITED, Mumbai (IN)

(21) Appl. No.: 13/375,686

(22) PCT Filed: Jun. 2, 2010
(86) PCT No.: PCT/IN10/00363

§ 371 Date: Feb. 6, 2012

(30) Foreign Application Priority Data
Jun. 2, 2009 (IN) 901/MUM/2009

(51) Int. Cl. C07D 401/12

(52) U.S. Cl. 546/273.7

(57) ABSTRACT

A process for the enantioselective synthesis of a sulphoxide of a compound of Formula I or a pharmaceutically acceptable salt thereof in the form of a single enantiomer or in an enantiomerically enriched form

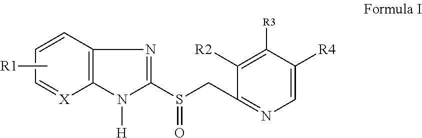

wherein R₁ to R₄ are same or different and selected from the group consisting of hydrogen, C₁ to C₄ linear or branched alkyl, C₁ to C₄ linear or branched alkoxy, aryl, aryloxy alkoxy substituted by halogen or alkoxyalkoxy; X is either CH or N,

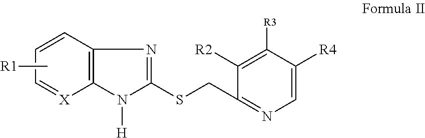

said process comprising oxidizing the prochiral sulphide, compound of Formula II in an organic solvent with an oxidizing agent in presence of titanium (IV)alkoxide, (−)-Diethyl-D-tartrate, C₁-C₄ alcohol, and water; and optionally converting the compound of formula I into a pharmaceutically acceptable salt.

### FIELD OF THE INVENTION

The present invention, relates to an improved process for producing sulphoxide compounds either as a single enantiomer or in an enantiomerically enriched form. More specifically, the present invention relates to a process for the enantioselective synthesis of substituted pyridinylmethyl sulfinyl-benzimidazoles of compound of Formula I,

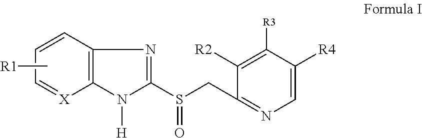

wherein R₁ to R₄ are same or different and selected from the group consisting of hydrogen, C₁ to C₄ linear or branched alkyl, C₁ to C₄ linear or branched alkoxy, aryl, aryloxy, alkoxy substituted by halogen or alkoxyalkoxy; X is either CH or N, by asymmetric oxidation of prochiral sulfide, compound of Formula II

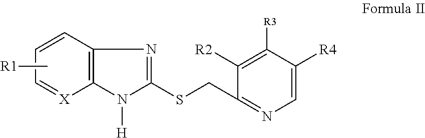

wherein R₁ to R₄ are as defined above, with an oxidizing agent in the presence of a chiral titanium complex, without using a base.

### BACKGROUND OF THE INVENTION

Various sulphoxide derivatives are known, and more particularly pyridinyl-methyl-sulfinyl benzimidazoles are known to be useful in therapeutics as, gastric acid secretion inhibitors. These compounds are also known as proton pump inhibitors. The first known derivative of the series of proton pump inhibitors is omeprazole, or 5-methoxy-2-[[(4-methoxy-3,5-dimethyl-2-pyridinyl)methyl]sulfinyl]-1H-benzimidazole which is useful as an antiulcer agent. Other derivatives of benzimidazole with similar structure are, lansoprazole, pantoprazole and rabeprazole. All these structurally related sulphoxide compounds have a stereogenic center at the sulphur atom and can exist as two enantiomers. It may be useful to separate them selectively under the form of one or the other of the two enantiomers with R and S configurations, or (+) and (−), whose specific properties can be different. It has been demonstrated that the (S) enantiomer of omeprazole, generically known as esomeprazole, shows improved physiological activity and pharmacokinetics as compared to the racemate of omeprazole. Esomeprazole is marketed in the form of a magnesium salt under the brand name Nexium®. Therefore, there is a need for a process for the manufacture of single enantiomer of pharmacologically active compounds.

Various methods have been described in the prior art to prepare single enantiomers of sulphoxide in a selective manner. These methods include enantioselective or chiral synthesis, optical resolution of a racemate, separating by converting the racemate to the diastereomers etc. The most attractive approach for obtaining a single enantiomer of sulphoxide is based on the enantioselective oxidation of sulfide. There are several methods known in the prior art, which discloses enantioselective synthesis of the single enantiomer of sulphoxide by the asymmetric oxidation of the sulfide.

U.S. Pat. No. 5,948,789, (herein after denoted as the '789 patent) describes an enantioselective synthesis of substituted sulphoxide by asymmetric oxidation of the prochiral sulfide. In this process, a prochiral sulphide is oxidized into the corresponding sulphoxide either as a single enantiomer or in an enantiomerically enriched form using an oxidizing agent in the presence of chiral titanium complex and in presence of base and organic solvent. The patent also discloses methods to achieve the sulphoxide in an enantiomerically enriched form in absence of a base. According to the '789 patent when a base is absent, the order of addition of components in to the reaction vessel should be altered and alternatively the time and/or temperature during the preparation of the chiral titanium complex is to be elevated. The preparation of the chiral titanium complex is preferably performed in presence of the prochiral sulfide and during an elevated temperature and a prolonged time when a base is not used in the reaction. Although the '789 patent suggests practicing these methods for carrying out the reaction in the absence of a base, it does not enable one to obtain the sulphoxide derivative in an enantiomerically enriched form as can be seen from the analytical results disclosed in Examples 6, 7, 8 and 14. It is clear that when the reaction is carried out in the absence of a base, the results are variable and the highest enantiomeric excess that was achieved was in Example 14 which discloses an enantiomeric excess of 87% only. Also reference examples A, B and C of '789 patent disclose that when the oxidation process of prochiral sulfide with titanium (IV) isopropoxide and diethyl tartrate is carried in the absence of a base and with different oxidizing agents and temperature conditions then the mixture obtained after the reaction contained high amounts of sulfide, sulfone and a mixture of enantiomers was obtained. When the present inventors carried out the reaction in the absence of a base at 40-45° C., it was observed that the reaction does not take place. (See Comparative Example 1) herein.

The recommended base in the '789 patent is an amine derivative namely Diisopropyl ethyl amine. A process which obviates the use of a base ensures that the final product is free from any trace level contamination of the base which is desirable since residual amine compounds in the end product need to be strictly restricted and controlled as per ICH guidelines to ppm/ppb levels as they may be toxic.

The '789 patent also provides an example wherein the base is absent and the reaction achieved by increasing the temperature and time which gives an amine free product, however the enantiomeric purity gets compromised by absence of base to an excess of 87% only, coupled with higher amounts of other impurities like the sulfide and sulfone which are at levels greater than that prescribed by ICH.

Also excessive temperature and reaction times are not desirable unless there are no alternatives. If it is possible to conduct the same reaction at a lower temperature and faster speed it saves on both energy requirements and processing times.

Thus there still remains an unmet need for an improved process for carrying out the oxidation of the prochiral sulphide which would result in the optically active sulphoxide in high enantiomeric excess, consistently. Also it would be preferable that the chiral titanium complex can be formed in lesser time and at lower temperatures. In addition to obtaining the sulphoxide derivative in an enantiomerically enriched form the other problems associated with the oxidation reaction such as formation of the corresponding sulfone derivative as an impurity and the presence of the unreacted sulfide in the final product may necessitate further purification by column chromatography or recrystallization. In view of this, a process for preparing an optically active sulphoxide in high optical purity, chemical purity and good yields are desired.

The present inventors have now found that an optically active sulphoxide and salts thereof can be prepared selectively with excellent enantiomeric excess in satisfactory yield by enantioselective oxidation of the corresponding prochiral sulfide in the presence of a chiral titanium complex without it being necessary to add a base. Also the process of the present invention obviates the need of using elevated temperatures for formation of the chiral titanium complex. Surprisingly, the present inventors have found that formation of the chiral titanium complex in the presence of a prochiral sulfide is facilitated by the presence of a C₁-C₄ alcohol. This followed by adding an oxidizing agent effects an unprecedented efficient chiral sulphoxide synthesis, under such conditions, resulting in the production of an optically active sulphoxide derivative having a high optical purity. Further, the use of C₁-C₄ alcohol surprisingly facilitates the chiral titanium complex formation in the presence of a prochiral sulfide at a temperature lower than 50° C. The process results in the production of a low level of sulfone impurities and lower residual sulfide starting materials. The product obtained, thus conforms to ICH standards.

### SUMMARY OF THE INVENTION

The present invention provides a process for the enantioselective synthesis of a sulphoxide of a compound of Formula I or a pharmaceutically acceptable salt thereof in the form of a single enantiomer or in an enantiomerically enriched form

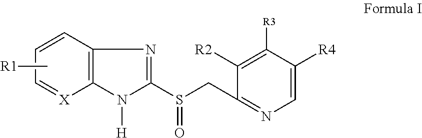

wherein R₁ to R₄ are same or different and selected from the group consisting of hydrogen, C₁ to C₄ linear or branched alkyl, C₁ to C₄ linear or branched alkoxy, aryl, aryloxy alkoxy substituted by halogen or alkoxyalkoxy; X is either CH or N,

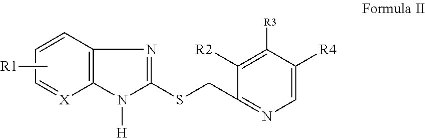

said process comprising oxidizing the prochiral sulphide, compound of formula II in an organic solvent with an oxidizing agent in presence of titanium (IV)alkoxide, (−)-Diethyl-D-tartrate, C₁-C₄ alcohol, and water; and optionally converting the compound of formula I into a pharmaceutically acceptable salt.

Thus the present invention does not require the presence of a base. In addition the elevated temperature and time required in prior art are significantly reduced by the addition of a C₁-C₄ alcohol which facilitates the reaction conditions and allows the reaction to proceed at a lower temperature and completes the same at a lesser time. It has been surprisingly found that the addition of a lower alcohol compensates the higher temperature and time required in the absence of a base and also results in a good enantiomeric excess especially for omeprazole. Earlier as reported in the '789 the absence of a base required one to raise the temperature and time for the reaction to proceed to completion and in addition ended up with an enantiomeric excess of a maximum of 87% only.

### DETAILED DESCRIPTION OF THE INVENTION

The present invention provides a process for enantioselective preparation of a sulphoxide of a compound of Formula I and their salts comprising asymmetric oxidation of the prochiral sulphide compound of Formula II with an oxidizing agent in an organic solvent in the absence of base, in presence of titanium (IV)alkoxide, (−)-Diethyl-D-tartrate, C₁-C₄ lower alcohol, and water. The titanium (IV) alkoxide is preferably titanium (IV) isopropoxide.

The C₁-C₄ alcohol is selected from the group consisting of methanol, ethanol and propanol. In one preferred embodiment C₁-C₄ alcohol is ethanol. The amount of ethanol used is 10% vol/wt with respect to the prochiral sulfide.

The amount of water used is 5% vol/wt with respect to the prochiral sulfide.

The organic solvent may be selected from the group consisting of toluene, xylene, tetrahydrofuran and the like. Preferably the organic solvent is toluene.

In one embodiment of the present invention the titanium (IV)alkoxide, and (−)-Diethyl-D-tartrate are mixed in an organic solvent followed by addition of the prochiral sulfide, C₁-C₄ alcohol and water at room temperature. The mixture thus obtained is heated in the temperature range of 40° C. to 45° C. The mixture is heated for 1.5 to 2 hours. The oxidizing agent is then added to the reaction mixture. Preferably the oxidizing agent is added after cooling the reaction mixture. After adding the oxidizing agent the temperature of the reaction mixture is maintained in the temperature range of −5 to 15° C. for a period of 1.0 to 1.5 hours. The oxidizing agent suitable for asymmetric oxidation may be an organic peroxide selected from hydrogen peroxide, alkylhydroperoxide such as tertiary butylhydroperoxide, and arylalkylhydroperoxides such as cumene hydroperoxide. Preferably the oxidizing agent is cumene hydroperoxide.

In one preferred embodiment the chiral titanium complex is prepared by mixing the (−)-Diethyl-D-tartrate and titanium (IV) isopropoxide in an organic solvent followed by addition of the prochiral sulfide, ethanol and water at room temperature. The reaction mixture is warmed to 40-45° C. and the prochiral sulfide gradually dissolves in the reaction system within a span of 1.5 to 2 hours resulting in a homogeneous reaction mixture. The reaction mixture is then cooled to 0 to −5° C. and cumene hydroperoxide is added. After addition of cumene hydroperoxide the temperature was raised in the range of 5 to 15° C. for a period of 1.0 to 1.5 hours

The resulting optically active sulphoxide compound prepared according to the present invention is further converted into alkali or alkaline earth metal salt of the sulphoxide by treating the optically active sulphoxide with an alkali or alkaline earth metal source. The alkali or alkaline earth metal source may be selected from bicarbonates, carbonates, hydrides, hydroxides such as sodium hydroxide, potassium hydroxide, magnesium hydroxide and the like. The alkali and alkaline earth metal salts of the optically active sulphoxide compound may be optionally converted to another alkali or alkaline earth metal salts.

In one preferred embodiment, the method of the present invention is used to oxidize the prochiral sulfide 5-Methoxy-2[((4-methoxy-3,5-dimethyl-2-pyridyl)methyl)-thio]-1H-benzimidazole, compound of Formula II wherein R₁ and R₃ are methoxy and R₂ and R₄ are methyl and X is CH to obtain selectively the (S) enantiomer of omeprazole, i.e., esomeprazole. Esomeprazole is obtained in excellent yields and purity. The esomeprazole obtained may be converted to its sodium salt which may be optionally converted to another alkali or alkaline earth metal salts. For example, esomeprazole sodium may be converted to esomeprazole magnesium.

The examples that follow do not limit the scope of the present invention and are included as illustrations.

### Example 1

### Preparation of Sodium (S)-5-Methoxy-2[((4-methoxy-3,5-dimethyl-2-pyridinyl)methyl) sulphinyl]-1H-benzimidazole

In a 3.0 lit RB flask was charged 400 ml of Toluene followed by 32 ml of (−)-Diethyl D tartrate and 28 ml Titanium isopropoxide and stirred to obtain a clear solution at 25-30° C., under nitrogen. To the above solution was added 100 g of 5-Methoxy-2[((4-methoxy-3,5-dimethyl-2-pyridyl)methyl)-thio]-1H-benzimidazole (Omeprazole sulfide) followed by 1.0 ml of ethanol and 1.0 ml of D.M. Water at 25-30° C. and stirred for 10-15 min. The reaction mixture was gradually heated to 43±2° C. internal temperature and was strictly maintained at 43±2° C. for 1.5 to 2 hours. The reaction mixture was cooled to 0 to −5° C. using ice water bath. To the cooled mixture was added cumene hydroperoxide (CHP, 70% aqueous solution) using addition funnel and temperature strictly maintained between 0 to −5° C. The reaction temperature was gradually raised to 10-15° C. and maintained for 80 to 90 minutes strictly at 10-15° C. To the reaction mixture, was added at 10-20° C., a solution of Sodium hydroxide in D.M. Water and stirring was continued for 15-20 min. at a temperature of 10-20° C. The aqueous layer was separated and washed with toluene. The aqueous layer was transferred to a 3.0 L 3 neck RBF equipped with overhead stirrer, thermometer pocket and nitrogen adapter. Methyl isobutyl ketone was added to the aqueous layer under stirring at temperature 25-30° C. The pH of the above mixture was adjusted using acetic acid to pH=7 to 7.8 and the reaction mixture was stirred for 15-20 min. at 25-35° C. The organic layer was separated. The aqueous layer was reextracted with MIBK and the pooled layers were dried over anhydrous sodium sulphate. To the MIBK solution was added 52.9 g of sodium methoxide solution in methanol (31% w/w) and stirred for 15 minutes at temperature 25-30° C. The solvents were distilled out from the above obtained clear solution on rotavapour at 45-50° C. under vacuum until the total volume was 300 to 400 ml. To the above mixture, acetonitrile was added at 25-30° C., under stirring. The obtained mass was stirred under nitrogen for 6-8 hr maintaining a temperature of 25-30° C. The product was filtered at 25-30° C., and washed with acetone and followed by acetonitrile and suck dried for 15-20 min. The product was dried on rotavapour under a vacuum at temperature 40-45° C., until the moisture content was less than 5%.

HPLC analysis: chiral purity: about 97.8%

Yield: 64.5 g

Sulfone: about 2.6

Sulfide: Not detected

### Example 2

### Preparation of Magnesium (S)-5-Methoxy-2[((4-methoxy-3,5-dimethyl-2-pyridinyl)methyl)sulphinyl]-1H-benzimidazole

Into a 2 L, 3N-RBF, at 25-28° C., was charged 700 ml of methanol followed by adding 150 g of sodium (S)-5-Methoxy-2[((4-methoxy-3,5-dimethyl-2-pyridinyl)methyl)sulphinyl]-1H-benzimidazole. The mixture was stirred for 10 minutes. The contents of the flask were cooled to 20-25° C. and stirred for 30 minutes at the same temperature. The solution was filtered through hyflo bed, and washed with methanol. The clear filtrate was collected and charged into a 2 L, 3N-RBF. To the above solution was added magnesium sulphate heptahydrate, in a single lot and stirred to obtain a suspension. The contents of the flask were stirred for 2 hours at 25-30° C. To the above mass/mixture.

Hyflo was charged and the mixture was further stirred for 10 minutes at 25-28° C. The contents of the flask were filtered through hyflo bed on buchner funnel and washed with methanol. The filtrate was collected and transferred the clear solution to 2.0 L R.B. flask assembly. The solvent was distilled out to a maximum temperature of 40-45° C. under vacuum. Acetone was charged and distilled out completely under vacuum. The mass was degassed for 30 minutes at 40-45° C. At 25-30° C., acetone was charged and stirred to obtain a white homogeneous slurry. The suspension was stirred for 2 hours at 25-30° C. The product was filtered at 25-30° C. and the product cake washed with acetone followed by D.M. Water. The product was suck dried to the maximum followed by drying in a vacuum oven at 40-45° C. until the moisture content was below 7%.

HPLC analysis: chiral purity: 99.8%

Sulfone: about 0.1° A

Yield: 100 g

### Example 3

Purification:

The obtained Esomeprazole Magnesium was further purified by following method. Into a 1 L, 3N-RBF, at 25-28° C., was charged 400 ml of methanol followed by adding 100 g of Esomeprazole Magnesium and stirring for 20 minutes. Charcoal was charged and stirred for 10 minutes. The solution was filtered through hyflo bed, and washed with methanol. The filtrate was collected and the clear solution was transferred to 1.0 L R.B. flask assembly. The solvent was distilled out to maximum at 40-45° C. under vacuum. Acetone was charged and distilled out completely under vacuum. The mass was degassed for 30 minutes at 40-45° C. At 25-30° C., acetone was charged and stirred to obtain a white homogeneous slurry. The suspension was stirred for 2 hours at 25-30° C. The product was filtered at 25-30° C. and the product cake washed with acetone followed by D.M. Water. The product was suck dried to the maximum followed by drying in a vacuum oven at 40-45° C. until the moisture content was below 7%.

HPLC analysis: chiral purity: 99.9%

Sulfone: Not detected

Yield: 80 g

### Comparative Example 1

### Preparation of Sodium (S)-5-Methoxy-2[((4-methoxy-3,5-dimethyl-2-pyridinyl)methyl)sulphinyl]-1H-benzimidazole

In a 3.0 lit RB flask was charged 400 ml of Toluene followed by 32 ml of (−)-Diethyl D tartrate and 28 ml Titanium isopropoxide and stirred to obtain a clear solution at 25-30° C., under nitrogen. To the above solution was added 100 g of 5-Methoxy-2[((4-methoxy-3,5-dimethyl-2-pyridyl)methyl)-thio]-1H-benzimidazole (Omeprazole sulfide) at 25-30° C. and stirred for 10-15 min. The reaction mixture was gradually heated to 50-55° C. internal temperature and maintained at the same temperature for about 1 hour. The reaction mixture was cooled to 0 to −5° C. using an ice water bath. To the cooled mixture was added cumene hydroperoxide (CHP, 70% aqueous solution) using addition funnel and temperature strictly maintained between 0 to −5° C. The reaction temperature was gradually raised to 10-15° C. and maintained for 80 to 90 minutes strictly at 10-15° C. HPLC analysis at this stage indicated no Esomeprazole formation.

Further investigation was conducted on the feasibility of using the above process which allowed for elimination of a base, lower reaction temperature and reaction times and use of C₁-C₄ alcohol to ascertain the applicability of the process to other benzimidazoles such as lansoprazole, pantoprazole and rabeprazole. It would be beneficial to the human race if this same process could yield an enantiomeric excess for all benzimidazoles coupled with low levels of sulfone and sulfide impurities. It is also known that the presence of these impurities in excess lead to physicochemical instability of the parent drug respectively.

Thus, lansoprazole sulfide, rabeprazole sulfide and pantoprazole sulfide were subjected to the above process wherein the base was absent and the process carried out at a lower temperature in the presence of a C₁-C₄ alcohol to ascertain whether one could obtain similar results for other benzimidazoles.

It was surprisingly observed that this process worked only for omeprazole and not for the others as depicted by the enantiomeric analysis content, the results of which are given in Table 1 below:

TABLE 1
Enantiomeric ratios of various benzimidazoles
by the process of the patent:

| S. No | Sulfides | Sulfoxide (Enantiomeric ratio) |
|---|---|---|
| 1 | Lansoprazole | Lansoprazole |
| | sulfide | (53:47) |
| 2 | Rabeprazole sulfide | Rabeprazole |
| | | (50:50) |
| 3 | Pantoprazole | Pantoprazole |
| | sulfide | (55:45) |
| 4 | Omeprazole sulfide | Omeprazole |
| | | (>99*) |

*(S)-isomer OR Esomeprazole

1. A process for the enantioselective synthesis of a sulphoxide of a compound of Formula I or a pharmaceutically acceptable salt thereof in the form of a single enantiomer or in an enantiomerically enriched form 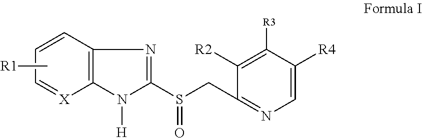
wherein R₁ and R₃ are methoxy, R₂ and R₄ are methyl and X is CH—, 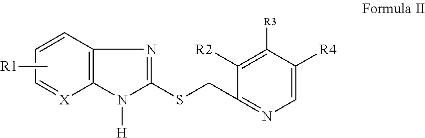
comprising oxidizing a prochiral sulphide compound of Formula II in an organic solvent with an oxidizing agent, in the absence of a base, in presence of a chiral titanium complex
wherein said chiral titanium complex is prepared by mixing titanium (IV)alkoxide and (−)-Diethyl-D-tartrate in the organic solvent, followed by addition of the prochiral sulphide, a C₁-C₄ alcohol, and -water;
and optionally converting the compound of Formula I into a pharmaceutically acceptable salt.

2. The process as claimed in claim 1, wherein said titanium (IV)alkoxide is -titanium (IV) isopropoxide.

3. The process as claimed in claim 1, wherein said C₁-C₄ alcohol is ethanol.

4. The process as claimed in claim 3, wherein a quantity of ethanol used is at least 10% volume by weight with respect to the prochiral sulfide.

5. The process as claimed in claim 1, wherein the oxidizing agent is cumene hydroperoxide or tertbutylhydroperoxide.

6. (canceled)

7. The process as claimed in claim 1 wherein the temperature required for the reaction is above 30° C. and the time required is at least 1.5 hours.

8. The process of claim 1, wherein a chiral purity of 97.8% or more is obtained.

9. A process for the enantioselective synthesis of a sulphoxide of compound of Formula I or a pharmaceutically acceptable salt thereof in the form of a single enantiomer or in an enantiomerically enriched form 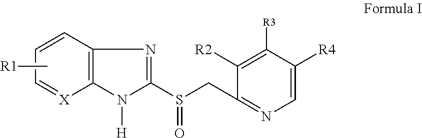
wherein R₁ and R₃ are methoxy, R₂ and R₄ are methyl and X is CH, 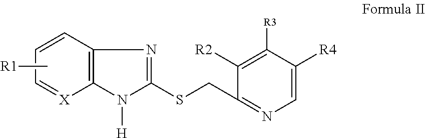
comprising oxidizing a prochiral sulphide compound of Formula II in an organic solvent in the absence of a base, in the presence of a chiral titanium complex, said process comprising the following steps in order
(1) mixing titanium (IV)alkoxide and (−)-Diethyl-D-tartrate in an organic solvent to form a chiral titanium complex;
(2) adding a prochiral sulphide compound of formula II, a C₁-C₄ alcohol, and water at room temperature;
(3) heating in a temperature range of 40° C. to 45° C. for 1.5 to 2 hours;
(4) adding an oxidizing agent; and
(5) optionally converting the compound of formula I into a pharmaceutically acceptable salt.
